(19) United States
(12) Patent Application Publication
(10) Pub. No.: US 2020/0405384 A1
(43) Pub. Date: Dec. 31, 2020

(54) DEVICES FOR TREATING LUNG TUMORS

(71) Applicant: ZIDAN MEDICAL, INC., New York, NY (US)

(72) Inventors: Dorin PANESCU, San Jose, CA (US); Mark GELFAND, New York, NY (US); Mark LEUNG, Duncan (CA)

(21) Appl. No.: 16/644,014

(22) PCT Filed: Sep. 7, 2018
(86) PCT No.: PCT/US2018/050023

§ 371 Date: Mar. 3, 2020

Related U.S. Application Data
(60) Provisional application No. 62/555,675, filed on Sep. 8, 2017.
(60) Provisional application No. 62/678,711, filed on May 31, 2018.

(51) Int. Cl. A61B 18/14; A61B 34/20; A61B 18/12; A61B 18/00

(52) U.S. Cl. CPC A61B 18/1492; A61B 34/20; A61B 2217/005; A61B 2034/2046; A61B 2018/1253; A61B 2018/00577; A61B 2018/00642; A61B 2018/144; A61B 2018/126; A61B 2018/00285; A61B 2018/1467; A61B 2018/00541; A61B 2218/002

(57) ABSTRACT

An apparatus (150) for ablating a tumor in a lung including: an elongate shaft (149); an obturator (152) positioned at a distal region of the elongate shaft, a suction lumen (166) extending through the elongate shaft and exiting the shaft distal to the obturator, wherein the suction lumen is configured to remove air or fluid from the portion of the lung, an ablation catheter delivery lumen (153) extending through the elongate shaft and exiting the shaft distal to the obturator, and an ablation catheter (155, 154) comprising an RF electrode (156, 157).

### RELATED APPLICATIONS

This application claims priority to U.S. provisional patent applications 62/555,675, filed Sep. 8, 2017, and 62/678,711, filed May 31, 2018, the entirety of both of these applications is incorporated by reference.

### TECHNICAL FIELD

The present disclosure is directed generally to devices and methods for ablating malignant lung tumors and more particularly to ablating lung tumors with an approach through the patient's airway.

### BACKGROUND

Lung cancer remains the leading cause of cancer-related deaths in the U.S. In fact, lung cancer is responsible for more deaths each year in this country than breast cancer, colon cancer, and prostate cancer combined. Non-small cell lung cancer (NSCLC) is the most common type of lung cancer; it is named for the type of cell within the lung where the cancer originates. Approximately 75 to 80% of individuals with lung cancer have NSCLC. Early NSCLC refers to cancer that has not spread widely outside of its site of origin. The earlier lung cancer is detected and treated, the better the outcome. The current standard treatment for early lung cancer consists of the surgical removal of as much of the cancer as possible followed by chemotherapy and/or radiation therapy.

Pneumonectomy or lobectomy (removal of a lung or lobe) with hilar and mediastinal lymph node sampling is the gold standard treatment for treating stage 1 or 2 non-small-cell-lung-cancer (NSCLC). Unfortunately, only about 15% to 30% of patients diagnosed with lung carcinoma each year are surgical candidates, either due to advanced disease or comorbidities. Particularly, many patients with concurrent Chronic Obstructive Pulmonary Disease (COPD) are not considered suitable for surgery.

Percutaneous pulmonary radiofrequency ablation (RFA) under CT guidance has become an increasingly adopted treatment option for primary and metastatic lung tumours. It is mainly performed in patients with unresectable or medically inoperable lung neoplasms. The immediate technical success rate is over 95%, with a low periprocedural mortality rate and 8 to 12% major complication rate. Pneumothorax represents the most frequent complication, but requires a chest tube drain in less than 10% of cases. Sustained complete tumour response has been reported in 85 to 90% of target lesions.

Bronchoscopic ablation of lung tumors is perceived by many as the next frontier in non-surgical thermal tumor ablation but has been held back by lack of specialized equipment for creation of large enough volume of destroyed tissue at the targeted site. This limitation is additionally challenged by the necessity to operate through the working channel of the bronchoscope and by the specific properties of lung tissue that is amply perfused, cooled by perfusion, evaporation and convection, and incorporates a large volume of air that can increase the volume of targeted tissue in phase with breathing. The latter consideration led to the lack of simple RF energy delivery bronchoscope-based instruments and preference was given to microwave energy, since microwave energy travels through air well. However, there is an advantage of simplicity and efficiency in RF heating of tissues that are appreciated in the field.

In light of the foregoing there remains a need for improvements to RF energy delivery methods and devices that prove suitability for bronchoscope-delivered ablation of lung tumors.

RF ablation may be utilized for treating various maladies, e.g., nodules of different organs like the liver, brain, heart, lung and kidney. When a nodule is found, for example within a lung, several factors are considered in making a diagnosis. For example, a biopsy of the nodule may be taken using a biopsy tool under CT guidance. Lately, biopsy tools have advanced the use bronchoscopy and allow a pulmonologist to obtain samples of tissue through airways. This procedure is known as transbronchial biopsy under fluoroscopic guidance, or under 3-D navigation using sensed tools, and its main limitation is inability to access smaller peripheral airways with a standard bronchoscope. Rapid miniaturization of such devices is a promising trend in the industry.

If the biopsy reveals that the nodule is malignant, it may prove useful to ablate the nodule. Under existing surgical scenarios, percutaneous treatment procedures can result in pneumothorax, which if not detected or repaired timely can ultimately lead to collapse of the lung. There is also considerable additional cost associated with CT guided interventional radiology procedures and often significant additional wait time for patients. Given the respiratory motion that occurs during breathing, transcutaneous approaches to the moving lung may impose a safety risk or difficulty precisely targeting a tumor. By approaching the peripheral lung targets through the bronchial tree, movement is less of an issue because the devices placed in the targeted airways, the parenchymal lung tissues and the targeted tumor move synchronously.

Endobronchial navigation uses CT image data to create a navigation plan to facilitate advancing an ablation catheter through a bronchoscope and a branch of the bronchus of a patient towards the nodule. Electromagnetic tracking may also be utilized in conjunction with the CT data to facilitate guiding the ablation catheter through the branch of the bronchus to the nodule. The ablation catheter may be positioned within one of the airways of the branched luminal networks adjacent to or within the nodule or point of interest. Once in position, fluoroscopy may be used to visualize the ablation catheter as it is further maneuvered towards the nodule or point of interest.

### SUMMARY

This disclosure is related to methods, devices, and systems for transbronchial ablation of a lung tumor. Aspects of the disclosure include:

Collapsing a portion of a lung comprising a tumor to ablate the tumor;

Compression of a portion of a lung comprising a tumor to ablate the tumor;

Surrounding a peripheral tumor with ablation electrodes;

Placing ablation catheters over guide wires and exchanging bronchoscope;

Ablating the tumor with RF ablation energy using bipolar, multi-polar and multiphasic RF configurations;

Ablating the tumor with RF ablation energy and irrigating the RF electrodes and controlling the RF ablation energy with temperature or impedance feedback;

Ablating the tumor with RF ablation energy using RF electrodes that are hydrophilic and relatively long for cooling tissue interface temperature and obtaining a more uniform current density profile;

Ablating the tumor with patterned lesions to overcome a need for accurate electrode navigation;

Placement of electrodes in airways using over the wire exchange of a bronchoscope and electrode catheter.

Exchanging a guided biopsy tool with a non-guided ablation tool upon a positive on-site biopsy result and maneuvering to the same biopsied location under fluoroscopy or ultrasound guidance; Decreasing blood flow to the targeted region of lung by decreasing oxygen in said region and causing local hypoxic vasoconstriction prior to or during delivery of ablation energy.

### BRIEF DESCRIPTION OF THE DRAWINGS

FIG. 1A is a schematic illustration of part of a human respiratory system.

FIG. 1B is a closer view of a section of FIG. 1.

FIG. 2 is a schematic illustration of multiple catheters positioned in a patient's airways to place energy delivery electrodes at different locations associated with a targeted tumor.

FIGS. 3A and 3B are schematic illustrations of distal region of an ablation catheter configured to deploy a pre-set shape to facilitate electrode apposition or fixation.

FIG. 4A is a schematic illustration of multiple RF electrodes positioned around a targeted tumor.

FIG. 4B is a schematic illustration of a cross section of FIG. 4A.

FIG. 4C is a plot of a multiphasic waveform.

FIG. 4D is a schematic of a multiphasic RF system.

FIG. 4E is a plot of a digital clock divided to generate a multiphasic RF configuration.

FIG. 5A is a schematic illustration if a distal region of a catheter configured to compress a portion of lung and ablate a tumor within the compressed portion.

FIG. 5B is a schematic illustration of the embodiment shown in FIG. 5A but in a compresses configuration.

FIG. 6 is a schematic illustration of a device that anchors to lung tissue and pulls on the anchors to compress tissue and ablate a tumor within the compressed tissue.

FIGS. 7A and 7B are schematic illustrations of an apparatus for collapsing a targeted segment of a lung and ablating a lung tumor.

### DETAILED DESCRIPTION

The present disclosure is directed generally to devices and methods for ablating malignant lung tumors and more particularly to ablating lung tumors with an approach through the patient's airway. An approach through the patient's airway may also be referred to as a transbronchial or endobronchial approach and comprises delivering medical devices through passageways by which air passes through the nose or mouth to the alveoli of the lungs. The term airway refers to any of the anatomical lumens of the respiratory system through which air passes including the trachea, bronchi, and bronchioles.

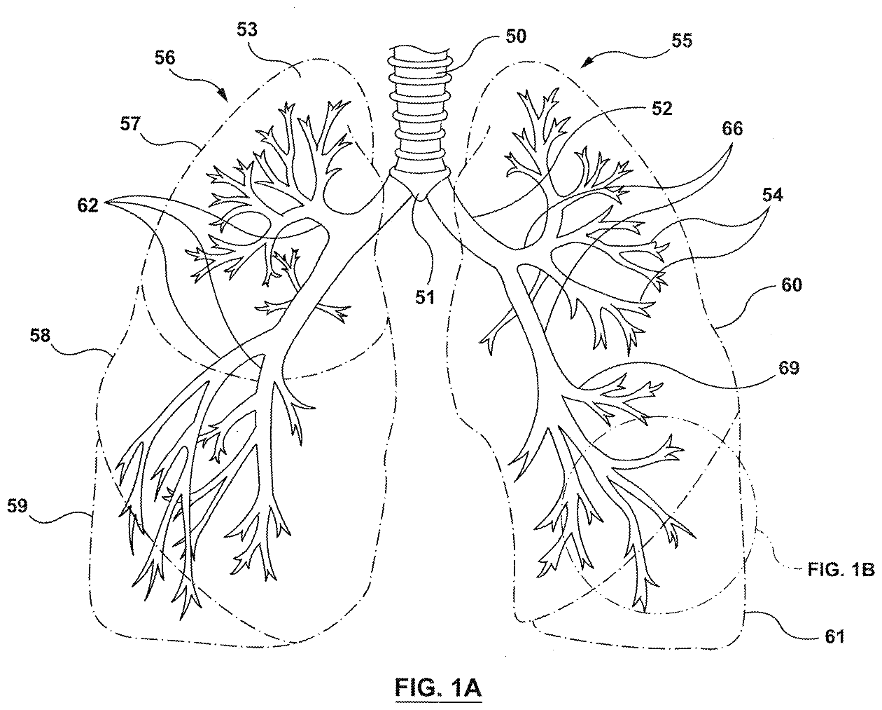

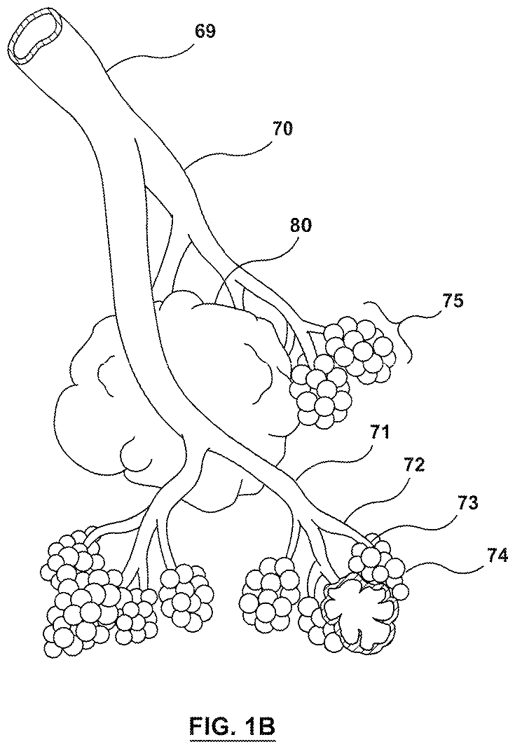

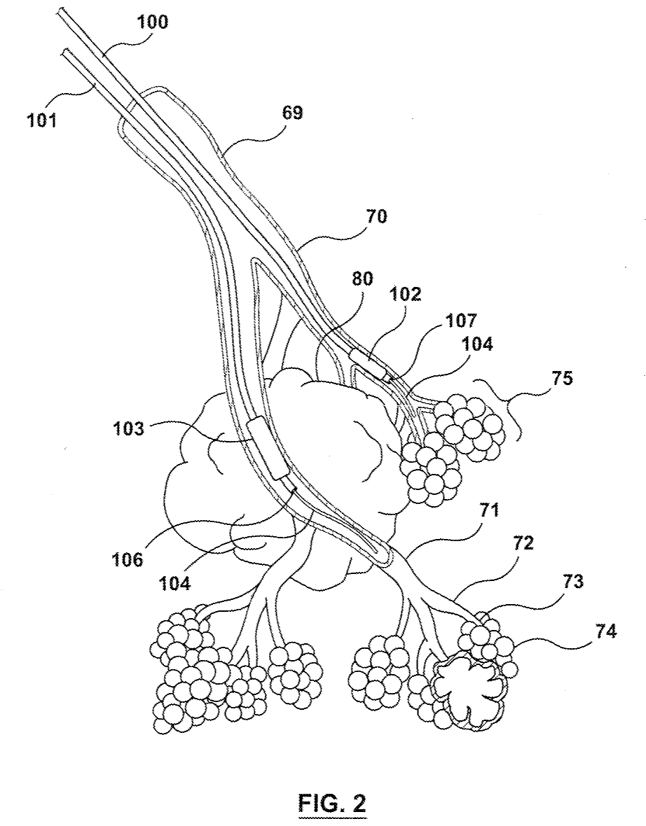

FIG. 1 is a schematic illustration of part of a patient's respiratory system including the trachea 50, carina of trachea 51, left main bronchus 52, right main bronchus 53, bronchioles 54, alveoli (not shown, residing in bunches at the end of bronchioles), left lung 55, right lung 56. The right main bronchus subdivides into three secondary bronchi 62 (also known as lobar bronchi), which deliver oxygen to the three lobes of the right lung—the superior lobe 57, middle lobe 58, and inferior lobe 59. The left main bronchus divides into two secondary 66 or lobar bronchi to deliver air to the two lobes of the left lung—the superior 60 and the inferior 61 lobes. The secondary bronchi divide further into tertiary bronchi 69, (also known as segmental bronchi), each of which supplies a bronchopulmonary segment. A bronchopulmonary segment is a division of a lung separated from the rest of the lung by a septum of connective tissue (not shown). As shown in FIG. 2 the tertiary bronchi 69 divide into many primary bronchioles 70, which divide into terminal bronchioles 71, each of which then gives rise to several respiratory bronchioles 72, which go on to divide into two to eleven alveolar ducts 73. There are five or six alveolar sacs 75 associated with each alveolar duct. Alveolar sacs are made up of several alveoli 74. The alveolus 74 is the basic anatomical unit of gas exchange in the lung. FIG. 2 also shows a peripherally located tumor 80 positioned in a space external to and amongst the bronchioles. A targeted tumor 80 may reside peripherally, centrally, or within a lymph node or airway wall of a lung or mediastinum.

There are two major types of lung cancer, non-small cell lung cancer (NSCLC) and small cell lung cancer (SCLC). Non-small cell lung cancer accounts for about 85 percent of lung cancers and includes: Adenocarcinoma, the most common form of lung cancer in the United States among both men and women, are formed from glandular structures in epithelial tissue and usually forms in peripheral areas of the lung; Squamous cell carcinoma, which accounts for 25 percent of all lung cancers and is more typically centrally located; Large cell carcinoma, which accounts for about 10 percent of NSCLC tumors. The focus of this disclosure is on treating NSCLC, which may occur peripherally among bronchioles, centrally among bronchi, or in lymph nodes.

An aspect of the disclosure provides a method for treating a lung tumor of a patient. A pathway to a point of interest in a lung of a patient is generated. An extended working channel is advanced through the airway into the lung and along the pathway to the point of interest. The extended working channel is positioned in a substantially fixed orientation at the point of interest. Anchoring mechanisms may be used to secure stability of the channel. A catheter may be advanced though the extended working channel to the point of interest. A working channel may be for example a lumen through a delivery sheath or through a bronchoscope, both of which may be steerable or incorporate a guidewire lumen. The lung tissue is treated with the ablation catheter at the point of interest. In the presented embodiments of this disclosure RF electrodes are used to deliver ablation energy.

An extended working channel may be positioned within a patient, optionally through a bronchoscope or as part of a bronchoscope. A locatable guide may be positioned within the extended working channel for positioning the extended working channel to the point of interest. Biopsy tools may be advanced to the point of interest. Prior to advancing the biopsy tool through the extended working channel, the locatable guide may be removed from the extended working channel. Alternatively, navigation-guided extended working channels may be used in conjunction with 3-D navigation systems, such those offered by Veran Medical or superDimension™ (Medtronic). The lung tissue may be biopsied. If the biopsy is confirmed positive, then the lung tissue may be ablated. The biopsy tool is retracted and replaced with an ablation catheter or tool comprising energy at least one delivery element. This method may facilitate positioning of the energy delivery elements of the ablation catheter or tool at the same place where the biopsy is taken. Prior to treating the lung tissue, the placement of the ablation catheter at the point of interest may be confirmed, for example visually using a bronchoscope and identifying the point of interest with respect to elements of the airway. The lung tissue or tumor may be penetrated at the point of interest. Effective treatment of the lung tissue may be confirmed.

With the current resolution of CT scanners, seven or eight generations of airways can be imaged and evaluated. There are reasons to believe that the imaging resolution will rapidly improve further. If the trachea is the beginning point and if a pulmonary parenchymal nodule is the targeted end-point, then appropriate software can interrogate the three-dimensional image data set and provide a pathway or several pathways through the adjacent airways to the target. The bronchoscopist can follow this pathway during a real bronchoscopy procedure and the correct airway pathway to the lesion can be quickly cannulated using a wire, a bronchoscope and a thin wall polymer tube or channel.

Once the access channel is in place, then multiple probes can be placed either to biopsy, or optically or by ultrasound testing the area of interest or to ablate the identified tumor. Ultrathin bronchoscopes can be used in a similar manner. Using these sorts of approaches, majority of peripheral lung lesions can be destroyed.

Currently available fiberoptic bronchoscopes (FOBs) have an illumination fiberoptic bundle and imaging fiberoptics or a camera. Except for the very few “ultrathin” bronchoscopes, there is also a channel for suction of secretions and blood, for the passage of topical medication and fluid for washing, and for the passage of various instruments for diagnostic retrieval of tissues or for therapeutic procedures. A typical diagnostic bronchoscope has an outer diameter of 5.0 to 5.5 mm and an operating channel of 2.0 to 2.2 mm. This caliber channel admits most cytology brushes, bronchial biopsy forceps, and transbronchial aspiration needles with sheathed outer diameters between 1.8 and 2.0 mm. Smaller bronchoscopes, in the range of 3.0 to 4.0 mm at the outer diameter and correspondingly smaller channels, are usually given a “P” designation (for pediatrics), but they can be used in the adult airways. Newer generations of slim video and fiberoptic bronchoscopes have a 2.0 mm operating channel with a 4.0 mm outer diameter. The one disadvantage of these bronchoscopes is the sacrifice of a smaller image area because of fewer optical bundles. The ultrathin bronchoscopes generally have outer diameters smaller than 3 mm. For example, Olympus models BF-XP40 and BF-XP160F (Olympus America, Center Valley, Pa.) have outer diameters of 2.8 mm and operating channels of 1.2 mm. Special instruments (e.g., reusable cytology brush and forceps) of the proper caliber are available for tissue sampling. Current generations of video bronchoscopes are all built with a 60 cm working length. These bronchoscopes are suitable for accessing distal airways to place the guide wire over which a delivery channel or an energy delivery catheter can be exchanged.

Surrounding the Tumor with Electrodes

A transbronchial lung tumor ablation procedure may comprise positioning multiple RF electrodes in airways surrounding a target ablation zone and delivering ablative RF energy from the RF electrodes to heat tissue in the target ablation zone wherein the target ablation zone comprises the tumor or is substantially comprised of the tumor and may furthermore comprise a margin of healthy tissue that may be ablated to ensure the compete tumor is ablated. A relatively small amount of non-tumorous tissue may be in the target ablation zone however it may be desired to ablate a minimal but safe amount of non-tumorous tissue to preserve lung function. Since lung tumors are not encapsulated, the ablation zone shall include margins that can be defined by clinician based on CT imaging. Current lung cancer ablation practitioners strive for 1 cm margins surrounding the targeted tumor nodule. RF energy may be delivered in various configurations to generate an ablative tissue temperature range (e.g., 55° C. to 100° C.) in the target ablation zone. For example, RF configurations may comprise multipolar (e.g., bipolar) mode or monopolar mode and may be multiphasic. Alternatively, RF energy elements may employ balloons. The balloons may be inflated with cooling fluid, such as physiological saline at temperature between 15 and 30° C., to protect adjacent tissue layers, such as mucosa and cartilages, from thermal damage. Energy may be delivered by means of electrodes mounted on the balloon surface, or electrodes located inside the balloon (capacitively or ohmically coupled to tissue), or through a balloon conductive wall when the balloon is inflated with a conductive fluid acting as an energy delivery element. A balloon may be sized to fit a targeted airway. A balloon may be used to place one or more ablation electrodes in to apposition with the airway wall to provide firm electrode contact.

For example, a monopolar RF energy delivery protocol may comprise the following parameters: power in a range of 1 to 50 W, duration for up to 300 seconds, a maximum tissue impedance of 1000 Ohms which may be used to terminate delivery of energy for safety and efficacy. Optionally, RF electrodes may be cooled for example with internal irrigation with a fluid such as sterile water or saline at a flow rate of up to 30 mL/min, which may allow delivery of higher power to achieve deeper lesions while avoiding overheating at the electrode-tissue interface. Optionally an ablation catheter may be equipped with 3D navigation sensors (e.g., electromagnetic, ultrasound, shape sensing) to facilitate guiding and delivery to a targeted location in the airways. In an embodiment that delivers monopolar RF energy in a long duration low power configuration may be advantageous to ablate at a relatively larger depth while avoiding over heating of tissue closer to the electrode. For example, such energy delivery parameters may comprise power in a range of less than 20 W, preferably less than 10 W, delivered in a range of up to 10 minutes, preferably less than 5 minutes. Both power and duration levels are chosen above levels required to cover location-specific thermodynamic conditions, such that sufficiently large lesions are produced. The RF electrodes may be equipped with temperature sensors, such that delivered powered is controlled according to set temperature targets. Set temperature values may be between 45° C. to 95° C., preferably between 50° C. to 80° C., depending on specific local conditions.

FIG. 2 shows two catheters 100 and 101 with energy delivery electrodes 102 and 103 as an example that can be introduced separately using a flexible bronchoscope and positioned with the electrodes terminating in two separate airways on two sides of the targeted tumor 80. The catheters may be delivered over a guide wire 104. The electrodes may be connected to electrical conductors that pass through the catheter shafts to a proximal region of the catheter for example terminating in an electrical connector, which may be electrically connected to an RF generator for example using a connector cable. Each catheter can incorporate more than one electrode that can be energized together or separately.

The electrodes of the catheters may be positioned at a desired location in an airway by delivering the catheters 100 and 101 over a guide wire 104 laid down for example using an ultrathin bronchoscope. Catheters 100 and 101 may comprise a guidewire lumen 106 and 107 and be adapted for over-the-wire (OTW) exchange. Currently available devices may be used to navigate to desired positions in the patient's airway. For example, electromagnetic navigation bronchoscopy is a medical procedure utilizing electromagnetic technology designed to localize and guide endoscopic tools or catheters through the bronchial pathways of the lung. Virtual Bronchoscopy (VB) is a three-dimensional, computer-generated technique that produces endobronchial images from spiral CT data. Using a virtual, three-dimensional bronchial map from a recently computed tomography (CT) chest scan and disposable catheter set, physicians can navigate to a desired location within the lung to biopsy lesions, take samples from lymph nodes, insert markers to guide radiotherapy or guide brachytherapy catheters. Such existing technology may be used to plan for a procedure, diagnose a tumor with a biopsy, or place a guidewire for positioning one or more treatment catheters. After a guide wire 104 is placed in an airway near the target ablation zone (e.g., within 0 to 10 mm from the target ablation zone or within the target ablation zone) the ultrathin bronchoscope can be withdrawn with the wire left in place and an electrode catheter may be exchanged over the wire.

Multiple catheters with electrodes, or balloon elements, can be placed in the described fashion by exchanging a bronchoscope for catheter over the wire. After the tumor is thus surrounded by energy delivery elements and the bronchoscope and guide wire are removed, the proximal ends of catheters can be connected to the RF generator outside of the body. The technology subject of the present disclosure can also be used to ablate lymph nodes, should biopsy results indicate lymph node metastases.

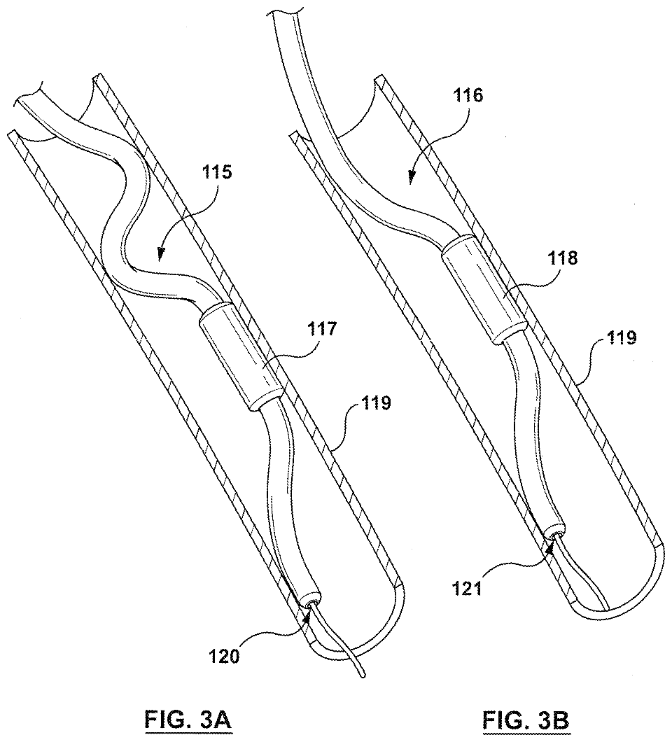

Radiopaque markers on the guide wire or catheter can be used to position the electrodes at the precise desired location. For example the RF electrodes may be radiopaque. Any of the ablation catheters disclosed herein may comprise a retention or anchoring mechanism at a distal region of the catheter to ensure its energy delivery element(s) stay in a desired position and avoid accidental dislodgement in particular when the patient breathes or coughs. For example, a retention or anchoring mechanism may comprise a section of the catheter that adopts a predefined non-linear shape as shown in FIGS. 3A and 3B, an inflatable balloon, spring loaded or wire activated splines, a stent, or deployable barbs positioned on the distal region of the catheter. Size and design of the electrode catheter can be made compatible with a working channel of regular or ultra thin bronchoscopes. Multiple electric connections for energy delivery and signal transmission (temperature and impedance) are envisioned. The ablation catheters may comprise a substance delivery lumen, which may be used to deliver substances into the airway such as drugs, contrast media to visualize the anatomy using fluoroscopy, and substances that induce lung collapse. Optionally, the guide wire lumen may function as the substance delivery lumen when the guide wire is removed, which may allow the catheter's diameter to be minimized. The ablation catheters may comprise an irrigation delivery lumen used to infuse irrigation fluid into the airway surrounding the electrodes to prevent charring and impedance rise and enable bigger lesion creation. The irrigation delivery lumen may be the same lumen as the substance delivery lumen or guide wire lumen.

Optionally, the distal region of the shaft of the ablation catheter may be configured to adopt a predefined non-linear shape such as a helix or arc that may be deployed when the distal region is positioned in a desired location in the airway to facilitate consistent electrode apposition with the wall of the airway and avoid intermittent apposition or axial movement of the electrodes. One embodiment as shown in FIG. 3A comprises a shaft section 115 in the distal region having a pre-set shape such as a helix while FIG. 3B comprises a shaft section 116 in the distal region having a pre-set shaped with an arc. For example the shaft section may be made from Pebax and molded to the desired pre-set shape. The shaft section may comprise a straightening wire that slidably engages a lumen 120 and 121 in the shaft section. The straightening wire may have a substantial stiffness to cause the shaft section to straighten when the wire is present in the lumen yet be flexible enough to allow the catheter to be advanced over a thin guide wire. Removing the straightening wire from the shaft section, for example retracting the wire by pulling on its proximal end from a proximal region of the catheter (e.g., on a handle), removes the straightening force exerted on the shaft section allowing it to elastically transform to its pre-set configuration. An electrode 117 and 118 is mounted on the shaped shaft section and is pressed against the wall 119 of the airway by the shaped shaft section.

Alternatively, one or more smaller diameter (e.g. 3 F to 6 F) catheters may be deployed through channels of a bronchoscope located at the proximal carina.

### Targeting Peripheral Tumors

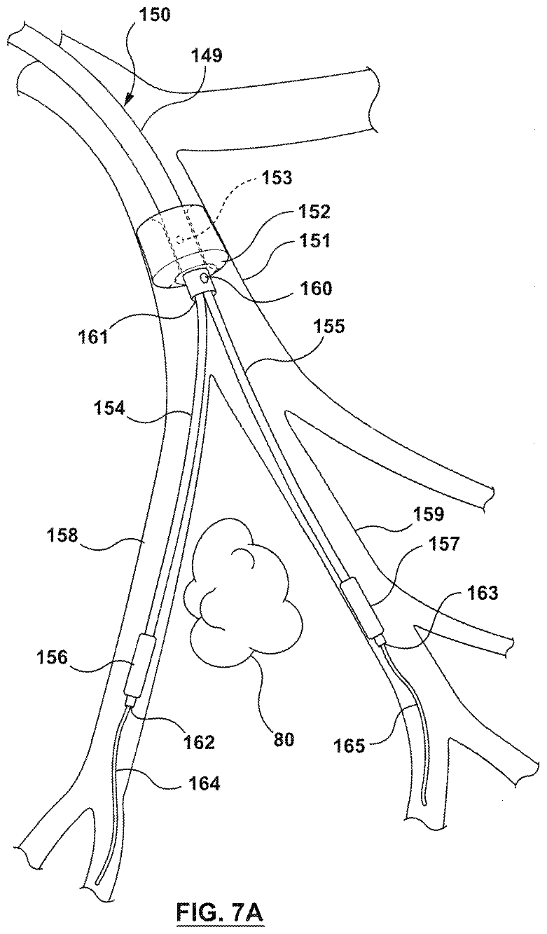

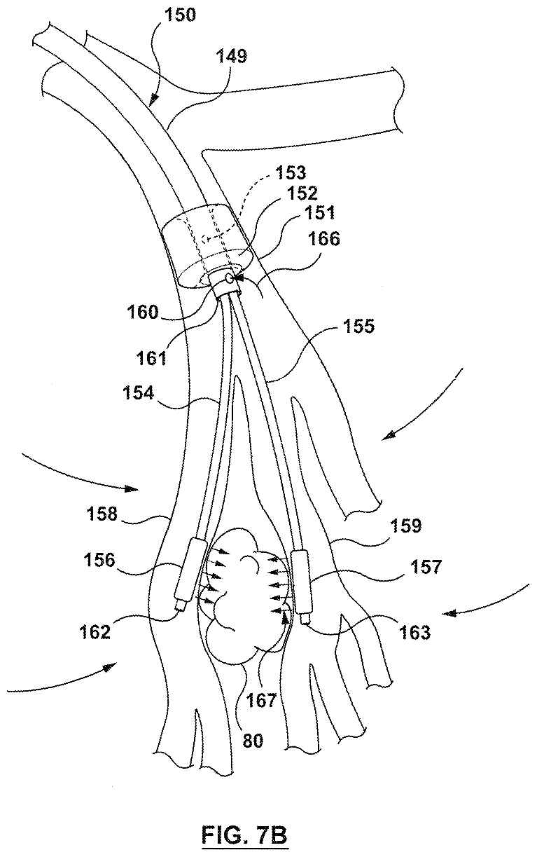

Access to tumors will vary greatly based on their location and size. Tumors located within two bifurcations from a main carina 51 (e.g., near a main bronchus 52 and 53, a secondary bronchus 62 and 66, or a tertiary bronchus 69) and adjacent to endo-bronchial surfaces are closer to reach than tumors near more distal airways and these closer airways have larger diameters. Tumors that are more challenging to reach may include tumors located distal to the third or fourth generations (e.g., near a tertiary or higher-generation bronchus or bronchiole). In an embodiment, a method of lung tumor ablation comprises collapsing the lung or portion of lung containing the targeted tumor if the targeted tumor is located peripherally (e.g., near a tertiary bronchus or bronchiole or past the third or fourth generations) but preserving the inflation of the lung when the targeted tumor is closer to main carina (e.g., near a main bronchus, secondary bronchus, or tertiary bronchus). Bronchial diameter decreases rapidly with distance away from main carina. Past the fifth generation of bifurcations, the average bronchus diameter decreases to less than 2 to 3 mm. As such, electrodes used in such areas may be less than or equal to 3 F to 5 F. Long, thin electrodes (e.g., diameter in a range of 0.5 to 2 mm and length in a range of 4 to 20 mm) may be placed in bronchi or bronchioles surrounding the targeted tumor and may be used for delivering energy to peripheral tumors. For example, a long, thin electrode may be flexible to navigate bends and may be a tightly wound coil. Given that such thin electrodes produce high current densities, to avoid unwanted effects of associated with high current density such as tissue charring, high tissue impedance, irregular or unpredictable energy delivery, the system may deliver relatively low power for long duration and may comprise monopolar RF delivery parameters as described above. Electrodes may be made of hydrophilic materials or comprise a hydrophilic coating to keep them wet to reduce tissue interface temperatures. Also, such materials have somewhat higher resistivities, which help make current density distributions more uniform, which may avoid or reduce hot spots. FIG. 7B shows electrodes 156 and 157 that may be long and thin with a hydrophilic coating (not shown) delivering RF ablation energy with an even current density along the length of the electrode (shown by field lines 167). For example, electrodes may be constructed with an electrically conductive cross-linked hydrophilic polymer coating disposed over at least a portion of the electrode. The cross-linked hydrophilic polymer coating includes a fibrous matrix comprising a plurality of discrete fibers and pores formed between at least a portion of the fibers and a hydrophilic polyethylene glycol-containing hydrogel network disposed within the pores of the fibrous matrix. Alternatively, electrodes 156 and 157 may be balloons inflated with cooling or non-cooling fluid. If inflated with cooling fluid, physiological saline at temperatures between 5 and 30° C. may be used. By cooling the balloon, adjacent tissues, such as mucosa and cartilage/collagen structures, may be protected from overheating and from thermal damage. In other embodiments, the balloons may be used to carry electrodes on their surface or inside their volumes. The balloon wall may offer capacitive or ohmic coupling to tissue. For example, thermoplastic polymers, such as varieties of polyethylene, may be employed to construct the balloon wall for general purposes or for balloons intended for providing capacitive coupling to tissue. For balloons intended to provide ohmic coupling to tissue, materials may be based on conductive polymers such as polypyrrole, polyaniline and poly(3,4-ethylenedioxythiophene). The size of such balloons is an important aspect. For ablating tumors, or metastatic lymph nodes, which require access via high-generation (e.g. 5th or greater) airways, use of balloon energy delivery elements provides an advantage given the balloon flexibility, collapsibility and easier conformance to any irregular aspects of the targeted airway shape.

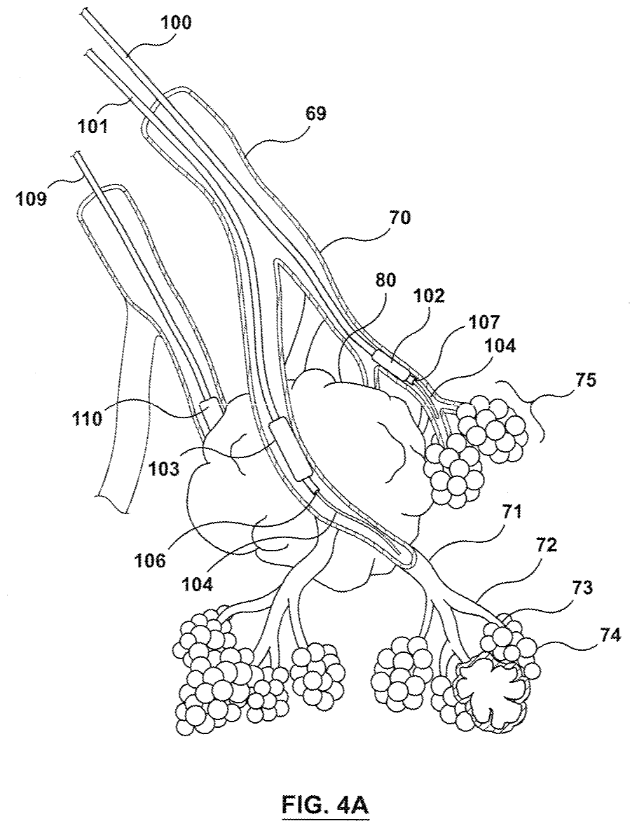

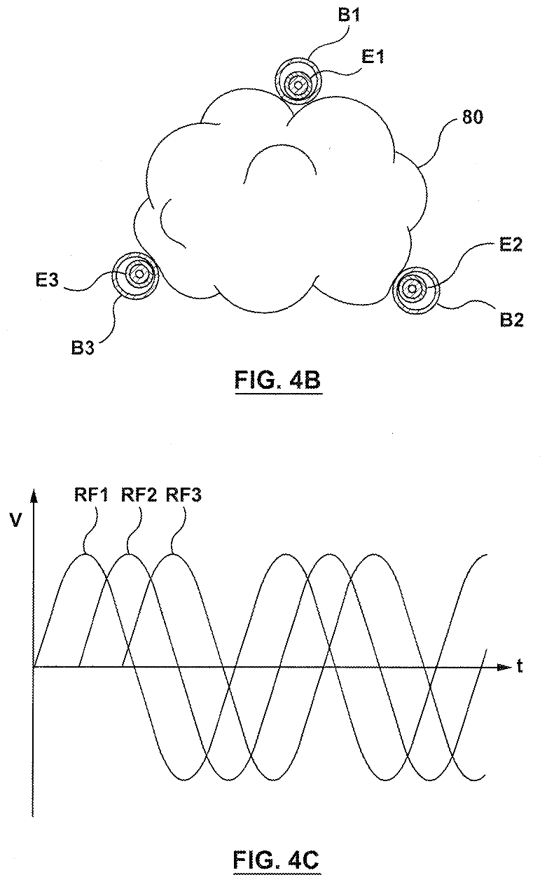

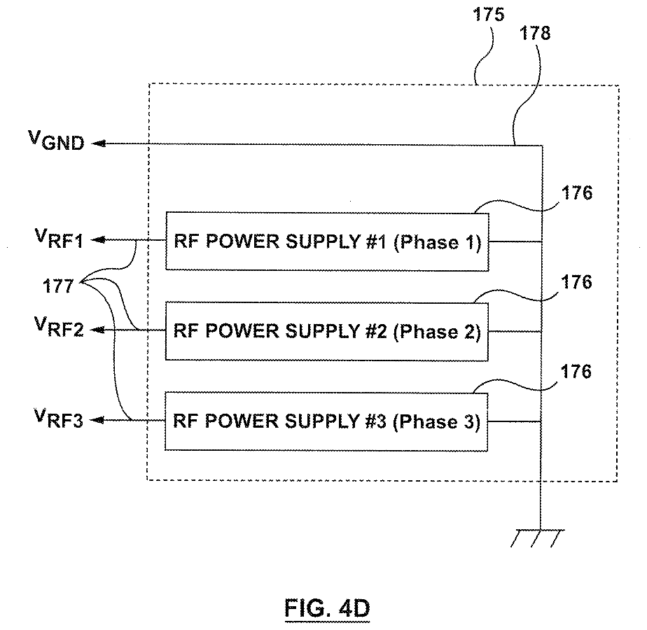

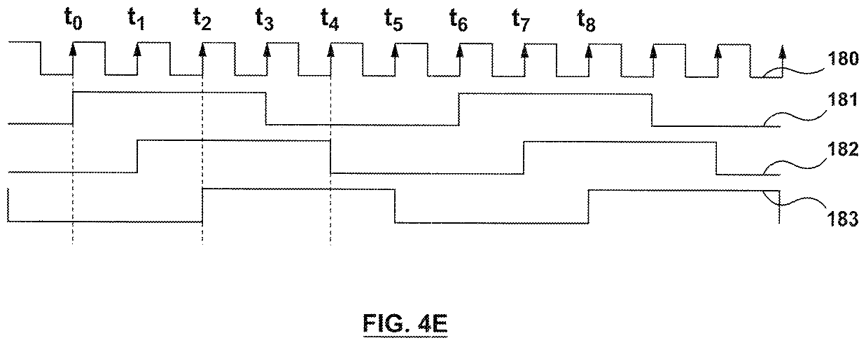

In an embodiment for ablating lung tumors (e.g., peripheral tumors), multi-electrode, or multi-balloon, multiphasic ablation concepts may be employed. FIG. 4A shows a tumor 80 surrounded by three electrodes, which in this illustration are delivered on three separate catheters 100, 101 and 109. More than three electrodes may be delivered without deviating from the concept. FIG. 4B is a cross sectional view of the targeted tumor 80 with three electrodes positioned around the tumor in adjacent airways. Thinner, longer electrodes may be deployed in airways surrounding the targeted peripheral tumor as described herein. Three RF electrodes labelled E1, E2 and E3 are positioned in three separate airways labeled B1, B2 and B3. For example, the three electrodes may be delivered on separate catheters, such as the catheter embodiment shown in FIG. 2. Multiphasic RF ablation waveforms may be used to set a rotating ablating electrical field, which delivers ablating energy to the tumor in a more localized modality. FIG. 4C illustrates a multiphasic RF waveform that may be used to ablate a targeted tumor encompassed by multiple RF electrodes, wherein RF1 is an RF signal delivered to electrode E1, RF2 is delivered to electrode E2, and RF3 is delivered to electrode E3. In this example, waveforms RF₁, RF₂ and RF₃ are 120° phase shifted apart. Application of such phased-shifted waveforms creates a rotating multipolar ablation field, which enhances the coverage of the tumor space and has the potential of providing more uniform lesions. In principle, phased RF ablation works similarly to bipolar ablation, except that electrical currents flow from or to a multitude of electrodes in a sequence dictated by phase differences. Each electrode is driven by an RF source having a different phase. The RF voltage resulting between each pair of electrodes (e.g., E₁-E₂, E₂-E₃ and E₃-E₁) drives RF current to flow in more uniform heating patterns in the tumor space. Power levels range between 1 to 20 W, with durations between 30 to 600 s. Temperature sensors may be employed with an intent to control local temperature values around a user-defined target. Temperature of such targets may vary in a range of 45 to 95° C., preferably in a range of 50 to 80° C. RF generators capable of delivering phased ablation energy may have additional RF output stages. FIG. 4D shows an example of a multiphasic RF energy console 175 providing an RF energy supply in which each output 177 has an independently controlled phase. The phase of RF signals at each output may be controlled by separate RF power supplies 176, or alternatively a central microcontroller, via software, or by hardware, for example by dividing a digital clock of a higher frequency, as shown in FIG. 4E. As shown in FIG. 4E a digital clock may comprise a base frequency 180 having a period (e.g., from t₀ to t₁) that is one sixth the period of frequencies 181, 182, and 183, which are delivered to the ablation electrodes and offset by one base period. Optionally, each electrode E₁, E₂, and E₃ (and respective RF output voltages V_{RF1}, V_{RF2} and V_{RF3}) may complete an electrical circuit with a dispersive ground pad connected to ground voltage V_{GND} at a terminal 178 of the RF energy supply 175. An alternative embodiment may comprise greater than three electrodes and waveforms.

An example of bipolar or multipolar RF ablation parameters that an RF console delivers to multiple electrodes, or to multiple balloons, or to combinations of balloon and electrode energy elements, may comprise power in a range of 1 to 50 W for a duration of 30 to 300 seconds. Tissue impedance may be expected to be in a range of 100 to 1000 ohms and the system may terminate or reduce power delivery if a high impedance (e.g., above 1000 ohms) is detected to avoid tissue char or uncontrolled ablation due to overheating, poor electrode contact with an airway wall. After desiccated tissue is rehydrated naturally or by irrigation, energy delivery can automatically resume. Impedance monitoring may also be used during energy delivery to determine if tissue temperature has raised sufficiently for an effective tumor ablation and instigate completion of energy delivery. The parameters may be used in a multiphasic RF ablation waveform or monophasic waveform. When balloon energy-delivery elements are employed, they may be inflated with cooling fluid, such as physiological saline at temperature between 15 to 30° C., to protect adjacent tissue layers, such as mucosa and cartilages, from thermal damage. Energy may be delivered by means of electrodes mounted on the balloon surface, or electrodes located inside the balloon (capacitively or ohmically coupled to tissue), or through a balloon conductive wall when the balloon is inflated with a conductive fluid acting as an energy delivery element.

### Compressing Lung Tissue During Tumor Ablation

In an embodiment for ablating a lung tumor, two or more bronchus branches containing a tumor or portion of a tumor between the branches may be drawn towards one another compressing the tissue between them. Energy delivery elements such as RF electrodes may be positioned in said branches. Compressing the tissue between the branches and energy delivery elements may facilitate ablation by concentrating ablative energy in the targeted tissue or bringing the electrodes closer to the tumor. The multiple RF electrodes may be configured in multipolar mode in which electrical current is passed between the electrodes as opposed to between one of the electrodes and a dispersive ground pad. The multipolar RF may comprise a multiphasic waveform. When electrodes are positioned close enough to one another it becomes possible to heat tissue between the electrodes to ablative temperatures due to the concentration of current density.

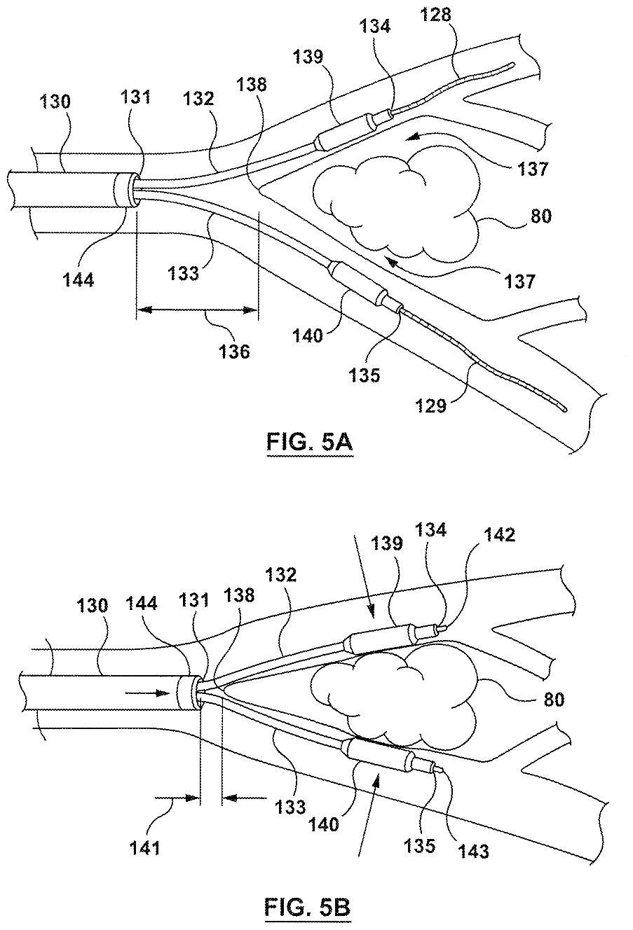

FIGS. 5A and 5B show a device that is inserted into the lung airways, for example using bronchoscope. It is equipped with prongs that can be introduced into a fork in the airway and activated to compress tissue caught between the prongs. The prongs can be equipped with RF electrodes in order to ablate the tissue between them. Multiple electrodes can be placed along the prongs. In the embodiment shown in FIG. 5A an elongate tubular sheath 130 comprises a lumen 131 through which two elongate prongs 132 and 133 are slidably engaged. An alternative embodiment may comprise more than two prongs without deviating from the concept. Electrodes 139 and 140 are mounted to the distal region of the prongs, wired through the prongs to an electrical connector on a proximal region of the device, and may comprise temperature sensors used to provide feedback to an energy delivery console. The electrodes may be configured according to various electrode embodiments disclosed herein such as long, thin electrodes, hydrophilically coated electrodes, and flexible electrodes. The prongs each comprise a lumen 134 and 135. The lumens 134 and 135 may be used to deliver the prongs over guide wires 128 and 129. The prongs may be made from an elastically deformable material such as Nitinol tubing and electrically insulated for example with an insulative coating which may also be lubricious to facilitate delivery through the lumen 131 and avoid short circuiting between electrodes 139 and 140. FIG. 5A shows the prongs delivered over guide wires 128 and 129 that in use are delivered through lumens 134 and 135 into targeted airway branches before the prongs are advanced out of lumen 131 and into the branches. As shown in FIG. 5A, prior to compressing the tissue and space 137 between the airway branches and the tumor 80 the sheath 130 may be positioned at a distance 136 (e.g., a minimum distance of about 1 inch) from the branch bifurcation 138. To apply a compressive force between the electrodes 139 and 140 the sheath 130 may be advanced toward the bifurcation 138 as shown in FIG. 5B. As the distance 141 decreases the bending radius of the prongs is decreased and due to the elastic nature of the prongs compression between the electrodes is increased. Optionally, additional compression may be generated by inserting stiffening mandrels 142 and 143 into the lumens 134 and 135 that increase the elasticity of the prongs. For example, stiffening wires 142 and 143 may be made of Nitinol wire or stainless steel and may extend the length of the device from the distal region to a proximal region. Optionally, stiffening wires 142 and 143 may be relatively flexible along their lengths to allow for delivery through a tortuous pathway and have a relatively stiff section that is advanced to the region of the prongs where they exit the sheath lumen 131 so stiffness is imparted only to the section of the prongs where increased stiffness will increase compression (e.g., a region from the electrodes to a position 1 or 2 inches in the sheath, which may be a length in a range of about 3″ to 5″). Optionally, the elongate sheath 130 may comprise a ring 144 of a rigid, strong material such as stainless steel mounted to the distal end of the elongate sheath to withstand the forces applied at the opening of the lumen 131 by the prongs 132 and 133. Ablation energy may be delivered through the electrodes 139 as described herein for example with a monopolar RF, multipolar RF, or in the case of more than two electrodes a multipolar multiphasic configuration. Multipolar or bipolar RF configuration may have an advantage of concentrating RF current density between the electrodes where the targeted tumor resides. In use the electrodes may be repositioned following an ablation to create a larger ablation, for example they may be slid back approximately one electrode length and ablation energy may be delivered again. To remove the device the sheath may be retracted to release compressive force between the electrodes and tissue and the prongs may be retracted into the sheath's lumen 131. The disclosure, as described above, can be applied to ablation of metastatic lymph nodes.

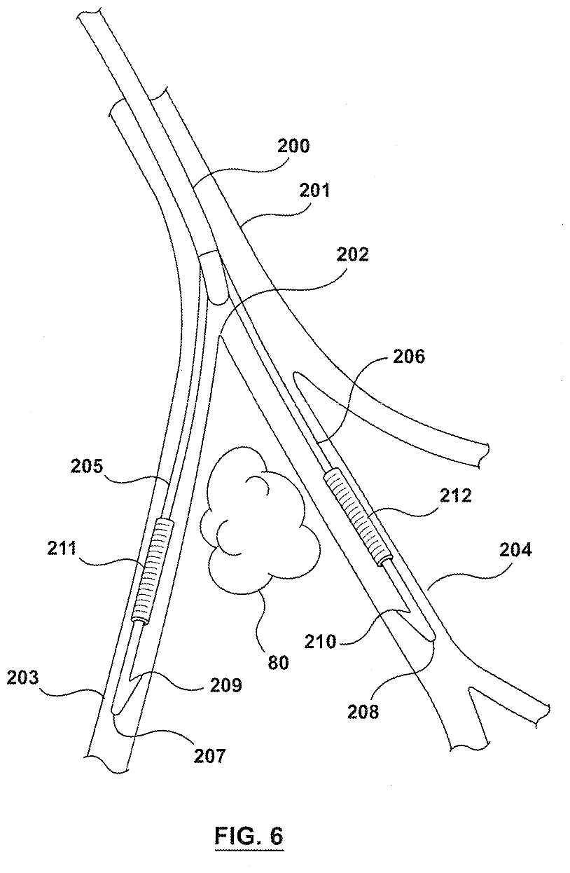

FIG. 6 shows an embodiment of a catheter configured to compress a portion of lung by pulling different branches of an airway toward a bifurcation and a target zone and ablate a tumor within the portion. A catheter 200 is shown placed in an airway 201 (e.g., a secondary bronchus) just proximal to a bifurcation 202 that branches into a first branch 203 and a second branch 204 (e.g., bronchioles). The catheter comprises at least two elongate arms 205 and 206 that are flexible and may be advanced into the branches 203 and 204. For example the arms 205 and 206 may travel through a lumen in the catheter 200 to the proximal end and a user may push the arms to deploy them from the catheter. The arms may comprise an electrically insulated Nitinol wire to facilitate pushability and flexibility. Each arm may comprise an anchor at its distal end to affix to tissue. For example, arms 205 and 206 may comprise a rounded tip 207 and 208 and a barb 209 and 210 that allows the arms to be advanced into the branches 203 and 204 and when pulled back the barbs engage the tissue and pull it back toward the bifurcation. The barbs may be designed to break off after an energetic pull on the catheter and remain embedded in the tissue. Alternatively, barbs may invert if pulled back forcefully and be dragged out of the airway. Retractable barbs can also be envisioned. The arms and barbs may be sized to be threaded through a peripheral airway such as a bronchiole (e.g., having an inner diameter, for example, of 2 mm, 1 mm, of 0.8 mm, of 0.6 mm, or 0.4 mm). Each arm may further comprise an energy delivery element such as an RF electrode 211 and 212. The electrodes may be long (e.g., 4 to 10 mm) and thin (e.g., 0.5 to 1 mm) and optionally may be flexible (e.g., made from a laser cut hypotube or tightly wound coil).

An alternative embodiment may be similar to the one shown in FIG. 6 however instead of barbs to grab tissue in separate branches of an airway prongs may comprise deployable anchors such as inflatable balloons or stents that may be deployed in position. The deployable anchor may generate sufficient friction to grab and pull on the airway branches to compress lung tissue and place electrodes mounted to the prongs closer to the targeted tumor. Following ablation, the deployable anchors may be retracted to release the tissue and the catheter may be removed from the lung.

### Collapsing a Portion of Targeted Lung Tissue

The lungs are divided into five lobes as shown in FIG. 1, including the right upper lobe, right middle lobe, right lower lobe, left upper lobe, and left lower lobe. The lobes are in tern divided into segments. Each lobe and segment is generally autonomous and receives its own bronchus and pulmonary artery branch. If an airway supplying a lobe or a segment is occluded with a one-way valve or occluded with an obturator and the air is sucked out it will collapse under the pressure exerted by the rest of the lung. Unlike most tissues in the body susceptible to tumors, lung tissue is intrinsically highly compliant, compressible and ultimately collapsible. Atelectasis refers to a complete or partial collapse of a lung, lobe or segment. When an airway is blocked, the blood absorbs the air inside the air sacs (alveoli). Without more air, the sac shrinks. The space where the lung was before the collapse fills up with blood cells, fluids and mucus. It is understood that in some cases collateral ventilation may re-inflate the collapsed segment but it is expected that tissue shrinking from building up heat and continuous suction can overcome, at least partially, the re-inflation of the target area.

Lung compliance is an important characteristic of the lung. Different pathologies affect compliance. Particularly relevant to cancer ablation are the observations that: fibrosis is associated with a decrease in pulmonary compliance; emphysema/COPD may be associated with an increase in pulmonary compliance due to the loss of alveolar and elastic tissue; and pulmonary surfactant increases compliance by decreasing the surface tension of water. The internal surface of the alveolus is covered with a thin coat of fluid. The water in this fluid has a high surface tension, and provides a force that could collapse the alveolus. The presence of surfactant in this fluid breaks up the surface tension of water, making it less likely that the alveolus can collapse inward. If the alveolus were to collapse, a great force would be required to open it, meaning that compliance would decrease drastically. Atelectasis is generally not desired. However, localized lung collapse can be beneficial in the treatment of emphysema and, as the authors propose, targeted lung cancer ablation. Advantages to collapsing the lung segment that contains a targeted tumor during tumor ablation may include the following: electrodes positioned in airways surrounding the tumor may be drawn closer to the tumor and improve concentration of ablative energy or increase efficacy of ablating the tumor; air will be removed from the collapsed airways and electrical and thermal impedance of tissue between the electrodes will be reduced; collapse of the segment may lead to hypoxia that provokes regional hypoxic pulmonary vasoconstriction of the lung segment which reduces metabolic cooling and improves efficient utilization of the thermal energy; and electrode contact with tissue may be more consistent or have greater surface area of contact, evaporation cooling and blood flow cooling may be reduced.

Collapsing of one lobe or a segment or other section of a lung defined by morphology of airways and air supply by airways can be impeded by collateral interlobular ventilation that is common in patients with incomplete interlobar fissures and partially damaged and destroyed lung. Alternative methods of segmental or lobar collapse can be employed by heating lung tissue or injecting chemicals, foam or hot steam into the targeted segment or the targeted lobe. For example injection of hot steam into a contained space like lobe or segment results in collapsing the space. The nature of the lung is such that when a segment is collapsed, pressurized adjacent segments compress it and fill the volume vacated by the collapsed space. Techniques for collapsing or partially collapsing portion of the lung that has collateral air pathways using a bronchoscope and bronchoscope delivered tools are described for example in U.S. Pat. No. 7,412,977 B2. Partial lung collapse, particularly of an upper lobe, was previously proposed to imitate results of lung reduction surgery in advanced emphysema but has not been suggested to enhance thermal ablation (e.g. RF) of tumors. Techniques proposed included: occluders and valves, steam (e.g., thermal), foam, and glue injection into airways. Mechanical compression of a lung portion using springs or wire coils was proposed also. All off these methods can be envisioned as being modified and adopted for cancer therapy in any lobe or segment where the tumor was located on CT and identified as malignant.

Ultimately an entire lung can be temporarily collapsed using a technique of independent lung ventilation. Lungs are intubated and ventilated by separate endotracheal tubes with obturators of the two main bronchi. An obturator is a device that obstructs an airway. Examples of obturators are balloons, valves, expandable meshes with a covering sheet and other such devices that are configured to be expanded to obstruct and airway and collapsed to be delivered to and removed from the airway. Balloons may be inflated and collapsed by delivery and suction of a fluid from an elongated shaft 149 that extends into the airway from the mouth of a patient.

A patient that is healthy enough to tolerate it can breathe using mechanical ventilation of only one lung while the contralateral lung is being collapsed and operated on. Electrodes can be positioned prior to deflating and collapsing the lung. In this case collateral ventilation will not have much effect on the ability of the operator to collapse the lung.

FIGS. 7A and 7B illustrate an ablation apparatus 150 introduced into a selected airway 151 comprising an elongated shaft 149, an obturator 152 positioned on a distal region of the shaft to occlude the airway, and a suction device (e.g., vacuum pump) to remove air from the airway 151 distal to the obturator 152 to collapse the targeted segment or lobe of the lung. Air may be removed from the targeted lung portion by applying negative pressure (e.g., with the suction device) to a lumen 160 positioned on the apparatus distal to the obturator 152, that pulls air from the lung portion through the lumen 160 to a proximal region of the apparatus 150 external to the patient. The elongated shaft 149 comprises a lumen 153 with a port positioned in the obturator for inflating and deflating the obturator. The shaft 149 further comprises lumen 161 through which one or more ablation catheters 154 and 155 may pass. The lumen 161 or obturator may comprise a one-way valve to impede external air from passing through the lumen 161 into the lung portion when the lung portion is under negative pressure and optionally allow air to escape. The ablation catheter(s) comprises at least one RF electrode 156 and 157 on each catheter, which are placed in branches 158 and 159 of the airway around a targeted tumor 80. The ablation catheters 158 and 159 may further comprise a guide wire lumen 162 and 163 so the catheters can be delivered over guide wires 164 and 165. As shown in FIG. 7B, when suction 166 is applied to remove air from the targeted lung portion the portion of lung is collapsed. Induced collapse of the lung segment or lobe brings the electrodes 156 and 157 close to the tumor 80 that, unlike the lung, is not easily compressible or collapsible. The obturator can include a one-way valve, allowing expiration but not inspiration, to facilitate collapse of the lung, lobe or segment supplied by the occluded airway.

### Blood Flow

Blood flow reduces efficiency of RF ablation by cooling tissue (i.e., removing energy). In practical terms, it means that higher blood flow per unit of volume of tissue limits the resulting lesion volume achieved per unit of energy delivered. Lungs are highly perfused.

Hypoxic pulmonary vasoconstriction (HPV) represents a fundamental difference between the pulmonary and systemic circulations. HPV is active in utero, reducing pulmonary blood flow, and in adults helps to match regional ventilation and perfusion although it has little effect in healthy lungs. HPV is a physiological phenomenon in which small pulmonary arteries constrict regionally in the presence of the regional alveolar hypoxia (low oxygen levels). Thus, reducing ventilation or oxygen supply of a lung region should also reduce perfusion of that segment via HPV.

Effects of blood flow and airway occlusion on the size of RF lesions in the lung has been investigated in literature but no practical solution has been proposed beyond balloon occlusion of the main bronchus and pulmonary artery (Anai Hiroshi et al. 2006. Effects of Blood Flow and/or Ventilation Restriction on Radiofrequency Coagulation Size in the Lung: An Experimental Study in Swine. Cardiol Intervent Radiol. 29(5):838-45). Such a method has limitations. Occlusion of the bronchus is compatible with percutaneous RF ablation but presents a challenge if a bronchoscope is used inside the same lung. Many patients with COPD will not be able to tolerate loss of the entire lung while under anaesthesia or after the procedure. This is one of the main reasons these patients are not considered surgical candidates.

Occluding an airway that ventilates the targeted lung segment and allowing it to become regionally hypoxic before energy application may by itself improve efficiency of any thermal ablation. It is expected that the blood flow will redistribute to other regions of the lung before the energy is applied.

This method can be further enhanced. In one embodiment, a gas that has low oxygen content such as a low-oxygen gas mixture or a gas such as nitrogen can be infused into the selected lobe or segment of the lung to replace oxygen temporarily to create hypoxia and induce regional HPV in the lobe or segment prior to or during delivery of ablation energy. For example, the embodiment shown in FIG. 7A previously described to suck air from a targeted portion of lung may be used to occlude a selected airway 151 with an obturator 152 positioned on an elongate shaft 149 and instead of removing air from the lung portion with a suction device, a low-oxygen gas may be infused through lumen 160 to induce HPV. One or more ablation catheters may be delivered through lumen 161 of the shaft 149. Optionally, air may be removed from the lung portion using a suction device connected to the lumen 160 and then the low-oxygen gas may be injected.

### Sequential Point Ablations

Reaching tumors less than 3 cm in size currently done under CT imaging guidance requires precise navigation of ablation electrode(s). As an alternative to delivering ablative energy under precise localization guidance, a series of point ablations may be delivered. For example, if the location of the tumor is only known with coarse precision (e.g., +/−2 cm) and if the tumor is, for example, 1 cm in diameter, point ablations can be performed at the nodes of a 5 cm by 5 cm grid with the nodes being 1 cm apart. This example requires 25 ablations but guarantees coverage of the tumor even if the navigation of the ablation catheter/instrument/electrode is imprecise. It is assumed that the ablation technology used in this example can deliver a lesion of 1 cm diameter. This may be done without compressing or collapsing a targeted portion of lung, in particular if the tumor is quite small and close to (e.g., within about 2 mm from the airway wall) or touching the airway wall. However, compressing or collapsing the lung may also be done along with sequential point ablations. Navigating a catheter through airways in a collapsed portion of lung to perform several point ablations may comprise positioning a catheter having multiple RF electrodes spaced apart according to the desired grid (e.g., 5 electrodes spaced apart every 1 cm on center) in an airway, then collapsing the portion of lung, then delivering RF ablation energy (e.g., discrete ablations from each electrode in monopolar mode, or multipolar mode). Collapsing or compressing the lung portion may have the benefit of improving electrode contact with airway wall, improving circumferential electrode contact and ablation around the airway, or bringing the targeted tumor closer to the electrodes. The portion of lung may be inflated after a set of ablation has been made and optionally the catheter may be moved to another location in the airway system within the targeted portion of lung to perform another set of ablations and the lung portion may be collapsed again. A device for collapsing a portion of the lung may be similar to the elongated sheath 149 with an obturator 152 shown in FIGS. 7A and 7B. However in this embodiment one or more ablation catheters may comprise a series of RF electrodes to cover a range of the desired grid.

1. An apparatus for ablating a tumor in a lung comprising:
an elongate shaft;
an obturator positioned at a distal region of the elongate shaft,
a suction lumen extending through the elongate shaft and exiting the shaft distal to the obturator, wherein the suction lumen is configured to remove air or fluid from the portion of the lung,
an ablation catheter delivery lumen extending through the elongate shaft and exiting the shaft distal to the obturator, and
an ablation catheter comprising an RF electrode.

2. The apparatus of claim 1 wherein the ablation catheter comprises a plurality of ablation catheters.

3. The apparatus of claim 2 wherein each of the plurality of ablation catheters are configured to be deployed simultaneously from the ablation catheter delivery lumen.

4. (canceled)

5. (canceled)

6. The apparatus of claim 1, further comprising an RF ablation console configured to deliver RF ablation energy to the RF electrode.

7. (canceled)

8. The apparatus of claim 1 wherein the RF ablation electrode(s) is configured to be delivered to a bronchiole and is at least one of a flexible electrode, a tightly wound wire coil, and a hydrophilic electrode.

9. (canceled)

10. The apparatus of claim 1, wherein the ablation catheter includes a three dimensional navigation sensor.

11.-26. (canceled)

27. A method of treating a patient with lung cancer comprising:
delivering a catheter through an airway of the patient to a target region in the patient's lung,
positioning the thermal energy delivery elements proximate to the tumor;
compressing a portion of the lung containing a tumor,
delivering ablative thermal energy to the compressed portion of the lung, and
ablating the tumor by the delivered ablative thermal energy.

28. The method of claim 27, wherein the compressing a portion of the lung comprises occluding an airway associated with the portion of lung and removing air from the portion of lung.

29.-54. (canceled)

55. A catheter no greater than three French in size and configured to ablate a tumor from within a bronchiole, the catheter comprising an elongated electrode having a diameter in a range of 0.5 to 1 mm and a length in a range of 4 to 10 mm.

56. The catheter of claim 55 further comprising a guidewire lumen.

57. The catheter of claim 55 further comprising an irrigation lumen.

58. The catheter of claim 55 further comprising a retention mechanism.

59. The catheter of claim 58, wherein the retention mechanism is an inflatable balloon, a distal section of catheter shaft configured to adopt a non-linear shape.

60. The catheter of claim 55 wherein the catheter is configured to be delivered through a working channel of a bronchoscope or an ultrathin bronchoscope.

61. The catheter of claim 55 wherein the electrode comprises an inflatable balloon.

62. The catheter of claim 55 wherein the electrode is flexible.

63. The catheter of claim 62 wherein the flexible electrode is a tightly wound wire coil.

64. The catheter of claim 55 wherein the electrode is hydrophilic.

65. The catheter of claim 64 wherein the electrode is coated with a cross-linked hydrophilic polymer comprising a fibrous matrix and a hydrophilic polyethylene glycol-containing hydrogel network disposed within the fibrous matrix.

66. The catheter of claim 55 further comprising multiple deployable arms each comprising an anchor on its distal end and an electrode.

67. The catheter of claim 66 wherein the anchor is a barb, balloon or stent.

68. The catheter of claim 66 wherein the deployable arms are slidably engaged in a lumen of the catheter, deploy from a distal region of the catheter up to 15 cm by pushing a proximal end of the arm from a proximal region of the catheter, and are retracted by pulling the proximal end of the arm.

69.-79. (canceled)
